(19) United States
(12) Patent Application Publication
(10) Pub. No.: US 2014/0039295 A1
(43) Pub. Date: Feb. 6, 2014

(54) PREMATURE NEONATE CRADLE

(71) Applicant: ASPECT IMAGING LTD., Shoham (IL)

(72) Inventor: Uri RAPOPORT, Moshav Ben Shemen (IL)

(21) Appl. No.: 13/906,785

(22) Filed: May 31, 2013

Related U.S. Application Data
(60) Provisional application No. 61/677,501, filed on Jul. 31, 2012.

(51) Int. Cl. A61G 11/00; A61M 16/08; A61B 5/055; A61M 16/00; A61M 16/12; A61M 1/00

(52) U.S. Cl. CPC A61G 11/00; A61M 16/12; A61M 1/00; A61B 5/0555; A61M 16/0057; A61M 16/0875
USPC 600/411; 600/22

(57) ABSTRACT

A cradle configured for life supporting a premature neonate during magnetic resonance imaging procedure. The cradle includes a receptacle configured for receiving the neonate and inserting into a MRI apparatus. The receptacle is magnetically transparent. The cradle includes a unit of neonate life support remotely connected to the receptacle such that a magnetic field of the MRI apparatus is not disturbed by the life support unit.

### FIELD OF THE INVENTION

The present invention relates to an apparatus for life support of a premature neonate and, more specifically, to a life support apparatus adapted for magnetic resonance imaging procedure.

### BACKGROUND OF THE INVENTION

Magnetic resonance imaging (MRI), nuclear magnetic resonance imaging (NMRI), or magnetic resonance tomography (MRT) is a medical imaging technique used in radiology to visualize internal structures of the body in detail. MRI makes use of the property of nuclear magnetic resonance (NMR) to image nuclei of atoms inside the body.

An MRI scanner is a device in which the patient lies within a large, powerful magnet where the magnetic field is used to align the magnetization of some atomic nuclei in the body, and radio frequency fields to systematically alter the alignment of this magnetization. This causes the nuclei to produce a rotating magnetic field detectable by the scanner—and this information is recorded to construct an image of the scanned area of the body. Magnetic field gradients cause nuclei at different locations to rotate at different speeds. By using gradients in different directions 2D images or 3D volumes can be obtained in any arbitrary orientation.

MRI provides good contrast between the different soft tissues of the body, which makes it especially useful in imaging the brain, muscles, the heart, and cancers compared with other medical imaging techniques such as computed tomography (CT) or X-rays. Unlike CT scans or traditional X-rays, MRI does not use ionizing radiation.

Life support in medicine is a broad term that applies to any therapy used to sustain a patient's life while they are critically ill or injured, as part of intensive-care medicine. There are many therapies and techniques that may be used by clinicians to achieve the goal of sustaining life. Some examples include feeding tube, Total parenteral nutrition, mechanical ventilation, heart/lung bypass, urinary catheterization, dialysis, cardiopulmonary resuscitation and other.

The ultimate goals of life support depend on the specific patient situation. Typically life support is used to sustain life while the underlying injury or illness is being treated or evaluated for prognosis. Life support techniques may also be used indefinitely if the underlying medical condition cannot be corrected but a reasonable quality of life can still be expected.

WO/2010/054457 ('457) discloses a transportable hospital device, such as an open type incubator, or a heated crib for intensive care or general use, particularly designed for the treatment of newborns, provided with at least one structure which is associated with at least one bed for the positioning of at least one patient. The equipment comprises: (i) at least one means for processing the operations operatively associated with the structure; (ii) at least one device for the treatment and life support of the patient integrated to the structure or to the bed; and (iii) at least one means for viewing information; the means for processing being configured to manage and process information about the patient supplied by at least one device for treatment and life support of the patient and the means for viewing the information being configured to make the processed information available.

The devices of life support known in the art (for example, as disclosed in '457) are characterized by a mass of metal articles. It is inadmissible to insert even parts of the known devices into a MRI apparatus. Thus, supporting life of a premature neonate cannot be performed in a ceaseless manner. There is a long-felt and unmet need to provide an device for neonate life support configured for inserting into the MRI apparatus such that a magnetic field created by the MRI apparatus is not disturbed by the neonate life support device.

### SUMMARY OF THE INVENTION

It is hence one object of the invention to disclose a portable stationary patient life support system comprising: a remote isolated specialist facility (RISF); a reversibly hermetically closable cradle providing an isolated environment, for use in the RISF; one or more stationary supply lines to stationary hospital services and utilities thereof; and, a portable life support unit (LSU) configured to continuously interconnect the portable supply lines and the cradle, such that the cradle is separable from the stationary supply lines whilst continuously and uninterruptedly non-interruptably connected to the portable LSU and the cradle is insertable into the RISF thereby ensuring that the patient is uninterruptedly connected to life support systems before, during and after transfer to RISF.

Another object of the invention is to disclose the portable stationary patient life support system as defined above, wherein the portable LSU is configured for maintaining a predetermined environment suitable for a patient

Another object of the invention is to disclose the portable stationary patient life support system as defined above, wherein the LSU is adapted to interface with stationary hospital services whilst maintaining the predetermined environment,

Another object of the invention is to disclose the portable stationary patient life support system as defined above, the LSU is further adapted to separate from the stationary hospital services and override them whilst maintaining the predetermined environment;

Another object of the invention is to disclose the portable stationary patient life support system as defined above, the LSU is additionally adapted to accompany a patient containing capsule up a proximity of an RISF and maintain the predetermined environment whilst the patient containing capsule is inserted into the RISF, and during sessions of the RISF and after a sessions in RISF.

Another object of the invention is to disclose the portable stationary patient life support system as defined above, the LSU comprises at least one source of medical gas.

Another object of the invention is to disclose the portable stationary patient life support system as defined above, the medical gas is selected from the group consisting of oxygen, compressed air, nitrous oxide, and carbon dioxide and any combination thereof.

Another object of the invention is to disclose the portable stationary patient life support system as defined above, the LSU comprises a suction unit.

Another object of the invention is to disclose the portable stationary patient life support system as defined above, the LSU comprises an electrical power supply.

Another object of the invention is to disclose the portable stationary patient life support system as defined above, wherein the LSU is configured to reversibly communicate with the capsule via an interface.

Another object of the invention is to disclose the portable stationary patient life support system as defined above, the LSU is connected to at least one adaptor insertable into a patient body.

Another object of the invention is to disclose the portable stationary patient life support system as defined above, wherein the adaptor is intravenously inserted into the patient's body.

Another object of the invention is to disclose the portable stationary patient life support system as defined above, wherein the adaptor is reliably mechanically fixated relative to the patient's body.

Another object of the invention is to disclose the portable stationary patient life support system as defined above, the system comprising a first environment provided by the hospitals stationary services encompassing the patient bed; a second environment for transporting the patient to an RISF; and a third environment embedded into the RISF.

Another object of the invention is to disclose a cradle configured for life support of a premature neonate during magnetic resonance imaging procedures; the cradle comprising a receptacle configured for receiving the neonate and inserting into an MRI apparatus; the receptacle being magnetically transparent; wherein the cradle comprises a neonate life support unit (LSU) remotely connected to the receptacle such that a magnetic field of the MRI apparatus is not disturbed by the LSU life support unit; thereby ensuring that the neonate is uninterruptedly connected to the LSU before, during and after inserting into an MRI apparatus transfer to RISF.

Another object of the invention is to disclose the cradle as defined above, wherein the life support unit is electric-grid independent.

Another object of the invention is to disclose the cradle as defined above, wherein the life support unit is battery energized.

Another object of the invention is to disclose the cradle as defined above, wherein the life support unit is configured for artificial pulmonary ventilation of the neonate.

Another object of the invention is to disclose the cradle as defined above, wherein the life support unit is configured for providing the neonate with maintenance media.

Another object of the invention is to disclose the cradle as defined above, wherein a breathing gas and the maintenance media are fed to the neonate by means of magnetically transparent feeding pipes.

Another object of the invention is to disclose the cradle as defined above, wherein the receptacle is provided with a connection interface device configured for interconnecting feeding pipes within the receptacle with the life support unit.

Another object of the invention is to disclose a patient-side immobilized system for stabilizing life supports service lines (LSSLs) to a patient in need of intensive care; the system comprising an autonomous cradle configured for life supporting a patient during treating in an RISF; the cradle comprising a receptacle configured for receiving the patient and inserting into the RISF; the receptacle being adapted to be compatible with the RISF; a receptacle adapted to be insertable within the RISF; a portable patient's life support unit (LSU), remotely connectable with the receptacle; wherein the LSU is reversibly interconnectable with (i) at least some of the stationary hospital services and (ii) with the receptacle thereby ensuring that the patient is uninterruptedly connected to the LSU before, during and after inserting into the RISF; and further wherein at patient's end the LSSLs are immobilizable thereby ensuring minimum trauma to the patient airways, blood vessels, circulatory system and/or gastroenterological tracts.

Another object of the invention is to disclose the system as defined above, wherein the system further comprises an LSSL-interface with LSSLs inlets on the exterior side of the interface and inside LSSL outlets on the interior side of the interface.

Another object of the invention is to disclose a method of treating a patient in need of intensive care; the method comprising the steps of: providing an autonomous cradle configured for life supporting a patient during treating in an RISF; the cradle comprising a receptacle configured for receiving the patient and inserting into the RISF; the receptacle being adapted to be compatible with the RISF; placing the patient into the receptacle; providing patient's life support by means of a portable LSU; remotely connected to the receptacle; inserting the receptacle into the RISF; and performing the treatment; wherein the LSU is reversibly interconnectable with (i) at least some of the stationary hospital services and (ii) with the receptacle; thereby ensuring that the patient is uninterruptedly connected to the LSU before, during and after inserting into the RISF.

Another object of the invention is to disclose the method as defined above wherein the method comprising the steps of providing an autonomous cradle configured for life supporting a premature neonate during magnetic resonance imaging procedure; the cradle comprising a receptacle configured for receiving the neonate and inserting into a MRI apparatus; the receptacle being magnetically transparent; placing the neonate into the receptacle; supporting neonate life performed by a portable unit of neonate life support unit (LSU) remotely connected to the receptacle such that a magnetic field of the MRI apparatus is not disturbed by the life support unit; inserting the receptacle into the MRI apparatus; performing MRI scan; wherein the LSU is reversibly interconnectable with (i) at least some of the stationary hospital services and (ii) with the receptacle; thereby ensuring that the neonate is uninterruptedly connected to the LSU before, during and after inserting into an MRI apparatus transfer to the MRI.

Another object of the invention is to disclose the method as defined above wherein the life support unit is electric grid independent.

Another object of the invention is to disclose the method as defined above wherein the life support unit is battery energized.

Another object of the invention is to disclose the method as defined above wherein the step of supporting neonate life comprises artificial pulmonary ventilation of the neonate.

Another object of the invention is to disclose the method as defined above wherein the step of supporting neonate life comprises administering maintenance media into the neonate.

Another object of the invention is to disclose the method as defined above wherein a breathing gas and the maintenance media are fed to the neonate by means of magnetically transparent feeding pipes.

Another object of the invention is to disclose the method as defined above wherein the receptacle is provided with a connection interface device configured for interconnecting feeding pipes within the receptacle with the life support unit.

Another object of the invention is to disclose a method of stabilizing life supports service lines (LSSLs) to a patient in need of intensive care; the method comprising the steps of: providing an autonomous cradle configured for life supporting a patient during treating in an RISF; the cradle comprising a receptacle configured for receiving the patient and inserting into the RISF; the receptacle being adapted to be compatible with the RISF; placing the patient into the receptacle; providing patient's life support by means of a portable LSU; remotely connecting the receptacle and LSSLs and immobilizing the same; inserting the receptacle into the RISF; and performing the treatment; wherein the LSU is reversibly interconnectable with (i) at least some of the stationary hospital services and (ii) with the receptacle; thereby ensuring that the patient is uninterruptedly connected to the LSU before, during and after inserting into the RISF; and further wherein at patient's end the LSSLs are immobilized thereby ensuring minimum trauma to the patient airways, blood vessels, circulatory system and/or gastroenterological tracts.

Another object of the invention is to disclose the method as defined above wherein the step of remotely connecting the receptacle and LSSLs and immobilizing the same further comprising step of providing an LSSL-interface with LSSLs inlets on the exterior side of the interface and inside LSSL outlets on the interior side of the interface.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIG. 1 is a schematic view of a cradle configured for life supporting a premature neonate during magnetic resonance imaging procedures;

FIG. 2 is a schematic view of a cradle inserted into a MRI apparatus; and

FIG. 3 is a schematic view of a cradle inserted into a MRI apparatus provided with a connection interface.

### DETAILED DESCRIPTION OF THE INVENTION

The following description is provided, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a cradle configured for life supporting a premature neonate during magnetic resonance imaging procedure. Additionally, a method of MRI a neonate demanding an intensive care is disclosed.

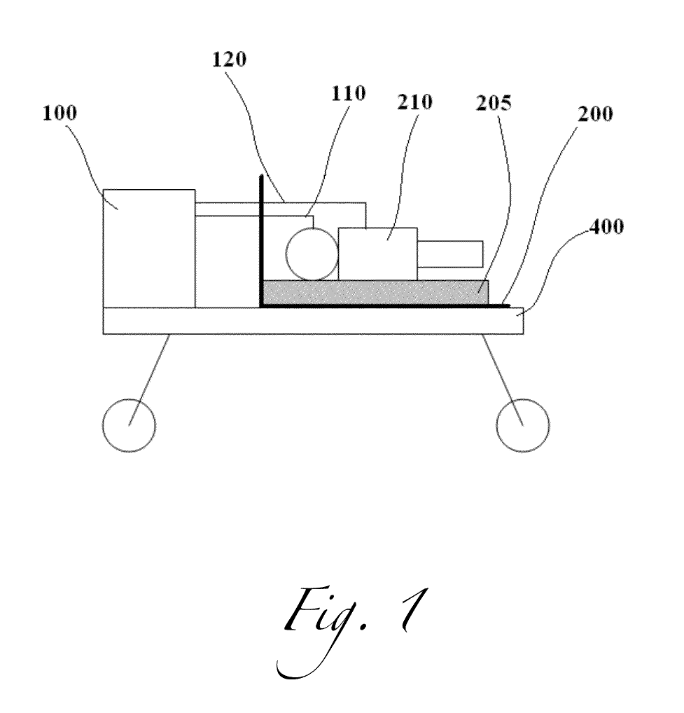

Reference is now made to FIG. 1, showing a cradle of the present invention. The afore the cradle comprises a receptacle 200 configured for receiving the neonate 210 and inserting into a MRI apparatus (not shown). The neonate is place on a support (cushion) 205. Vital activity of the neonate 210 is supported by a life support unit 100. The aforethe unit 100 provides artificial pulmonary ventilation for the neonate 210. Additionally, maintenance media are also provided to the neonate 210. Vital gas, fluid and maintenance media are fed through pipes 110 and 120, respectfully. It should be emphasized that the receptacle 200, the support 205 and the pipes 110/120 are magnetically transparent and do not dither a magnetic field created the MRI apparatus. The life support unit 100 is remotely connected to the receptacle such that a magnetic field of the MRI apparatus is not also disturbed by the life support unit. The disclosed cradle is easily transportable because all components thereof are mounted on a trolley 400. The life support unit 100 can be electric grid independent. Specifically, the life support unit 100 can be battery energized.

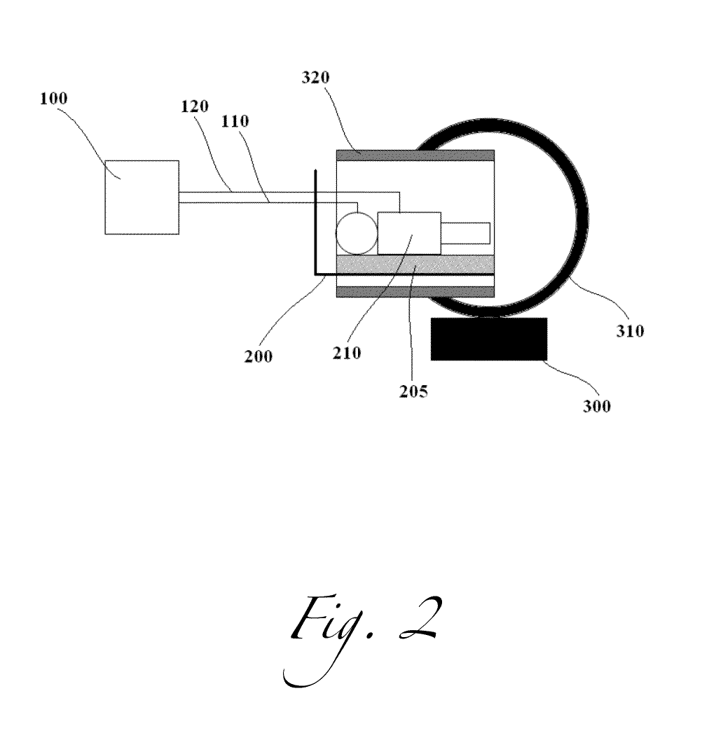

Reference is now made to FIG. 2, presenting the receptacle 200 inserted into the magnet 320 held by an arc 310 mounted onto a abut 300. The neonate 210 is within a magnetic field created by magnet 320. Results of performed MRI scans are not disturbed by cradle components because the receptacle 200, the support 205 and the pipes 110/120 are made of non-magnetic materials. Additionally, the life support unit 100 which can comprise articles made of magnetic material is remotely placed out such that the unit 100 does not distort the aforethe field.

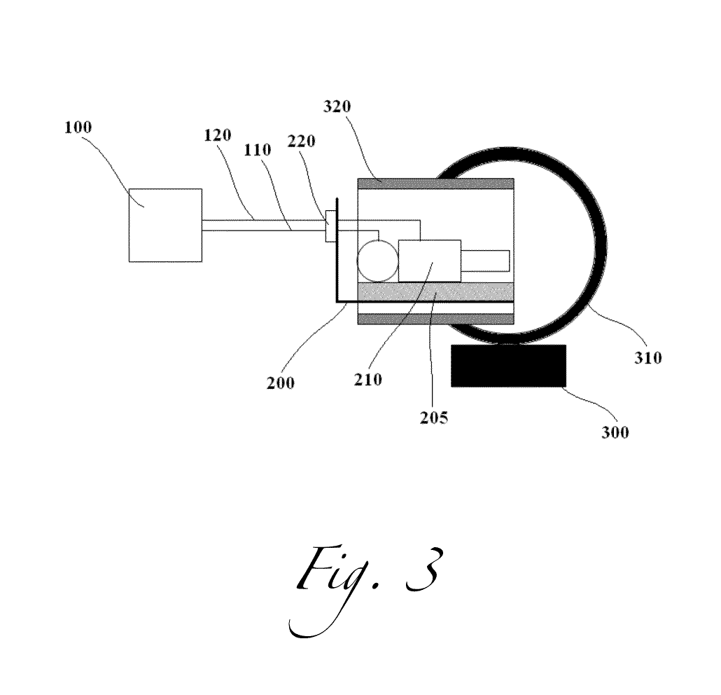

Reference is now made to FIG. 3, showing an alternative embodiment of the present invention. As differentiated from the embodiment depicted in FIG. 2, a connection interface 220 is provided. The interface 220 is designed for easy connection/disconnection of the life support unit 100 and replacement thereof. Specifically, life support unit 100 can be easily replaced with a life support unit energized by means of electric grid of a hospital (not shown).

According to the present invention, a cradle configured for life supporting a premature neonate during magnetic resonance imaging procedure is disclosed. The aforethe cradle comprises a receptacle configured for receiving the neonate and inserting into a MRI apparatus. The receptacle is magnetically transparent.

It is a core feature of the invention to provide the cradle comprising a unit of neonate life support remotely connected to the receptacle such that a magnetic field of the MRI apparatus is not disturbed by the life support unit.

According to one embodiment of the present invention, the life support unit is electric grid independent.

According to another embodiment of the present invention, the life support unit is battery energized.

According to a further embodiment of the present invention, the life support unit is configured for artificial pulmonary ventilation of the neonate.

According to a further embodiment of the present invention, the life support unit is configured for providing the neonate with maintenance media.

According to a further embodiment of the present invention, a breathing gas and the maintenance media are fed to the neonate by means of magnetically transparent feeding pipes.

According to a further embodiment of the present invention, the receptacle is provided with a connection interface device configured for interconnecting feeding pipes within the receptacle with the life support unit.

According to a further embodiment of the present invention, a method of MRI a neonate demanding an intensive care is disclosed. The aforethe method comprising the steps of: (a) providing an autonomous cradle configured for life supporting a premature neonate during magnetic resonance imaging procedure; the cradle comprising a receptacle configured for receiving the neonate and inserting into a MRI apparatus; the receptacle being magnetically transparent; (b) placing the neonate into the receptacle; (c) supporting neonate life; (d) inserting the receptacle into the MRI apparatus, (e) performing MRI scan; and (f) removing the neonate from the receptacle.

It is another core feature of the invention to provide the step of supporting neonate life performed by a unit of neonate life support remotely connected to the receptacle such that a magnetic field of the MRI apparatus is not disturbed by the life support unit.

According to a further embodiment of the present invention, the step of supporting neonate life comprises artificial pulmonary ventilation of the neonate.

According to a further embodiment of the present invention, the step of supporting neonate life comprises administering maintenance media into the neonate.

What is claimed is:

1. A stationary patient life support system comprising:
(a) a remote isolated specialist facility (RISF); a
(b) a reversibly hermetically closable cradle providing an isolated environment, for use in the RISF;
(c) stationary supply lines to stationary hospital services and utilities thereof; and,
(d) a portable life support unit (LSU) configured to continuously interconnect the portable supply lines and the cradle, such that the cradle is separable from the stationary supply lines whilst continuously and uninterruptedly non-interruptably connected to the portable LSU and the cradle is insertable into the RISF thereby ensuring that the patient is uninterruptedly connected to life support systems before, during and after transfer to RISF.

2. The stationary patient life support system according to claim 1, wherein at least one of the following is being held true (a) the portable LSU is configured for maintaining a predetermined environment suitable for a patient; (b) the LSU is additionally adapted to accompany a patient containing capsule up a proximity of an RISF and maintain the predetermined environment whilst the patient containing capsule is inserted into the RISF, and during sessions of the RISF and after a sessions in RISF; (c) the LSU comprises at least one source of medical gas; (d) the medical gas is selected from the group consisting of oxygen, compressed air, nitrous oxide, and carbon dioxide and any combination thereof; (e) the LSU comprises a suction unit; and any combination thereof.

3. The stationary patient life support system according to claim 1, wherein the LSU is adapted to at least one selected from a group consisting of (a) interface with stationary hospital services whilst maintaining the predetermined environment; (b) separate from the stationary hospital services and override them whilst maintaining the predetermined environment; and any combination thereof.

4. The stationary patient life support system according to claim 1, wherein at least one of the following is being held true (a) the LSU comprises an electrical power supply; (b) the LSU is configured to reversibly communicate with the capsule via an interface; (c) the LSU is connected to at least one adaptor insertable into a patient body; (d) the adaptor is intravenously inserted into the patient's body; (e) the adaptor is reliably mechanically fixated relative to the patient's body; and any combination thereof.

5. The stationary patient life support system according to claim 1, comprising
a. a first environment provided by the hospitals stationary services encompassing the patient bed;
b. a second environment for transporting the patient to an RISF; and
c. a third environment embedded into the RISF.

6. A cradle configured for life support of a premature neonate during magnetic resonance imaging procedures; the cradle comprising a receptacle configured for receiving the neonate and inserting into an MRI apparatus; the receptacle being magnetically transparent; wherein the cradle comprises a neonate life support unit (LSU) remotely connected to the receptacle such that a magnetic field of the MRI apparatus is not disturbed by the LSU life support unit; thereby ensuring that the neonate is uninterruptedly connected to the LSU before, during and after inserting into an MRI apparatus transfer to RISF.

7. The cradle according to claim 6, wherein the life support unit is electric-grid independent.

8. The cradle according to claim 7, wherein the life support unit is battery energized.

9. The cradle according to claim 6, wherein the life support unit is configured for artificial pulmonary ventilation of the neonate.

10. The cradle according to claim 6, wherein the life support unit is configured for providing the neonate with maintenance media.

11. The cradle according to claim 6, wherein a breathing gas and the maintenance media are fed to the neonate by means of magnetically transparent feeding pipes.

12. The cradle according to claim 6, wherein the receptacle is provided with a connection interface device configured for interconnecting feeding pipes within the receptacle with the life support unit.

13. A patient-side immobilized system for stabilizing life supports service lines (LSSLs) to a patient in need of intensive care; the system comprising
a. an autonomous cradle configured for life supporting a patient during treating in an RISF; the cradle comprising a receptacle configured for receiving the patient and inserting into the RISF; the receptacle being adapted to be compatible with the RISF;
b. a receptacle adapted to be insertable within the RISF;
c. a portable patient's life support unit (LSU), remotely connectable with the receptacle;
wherein the LSU is reversibly interconnectable with (i) at least some of the stationary hospital services and (ii) with the receptacle thereby ensuring that the patient is uninterruptedly connected to the LSU before, during and after inserting into the RISF; and
further wherein at patient's end the LSSLs are immobilizable thereby ensuring minimum trauma to the patient airways, blood vessels, circulatory system and/or gastroenterological tracts.

14. The system of claim 13, wherein the system further comprises an LSSL-interface with LSSLs inlets on the exterior side of the interface and inside LSSL outlets on the interior side of the interface.

15. A method of treating a patient in need of intensive care; the method comprising the steps of:
a. providing an autonomous cradle configured for life supporting a patient during treating in an RISF; the cradle comprising a receptacle configured for receiving the patient and inserting into the RISF; the receptacle being adapted to be compatible with the RISF;
b. placing the patient into the receptacle;
c. providing patient's life support by means of a portable LSU; remotely connected to the receptacle;
d. inserting the receptacle into the RISF; and
e. performing the treatment;
wherein the LSU is reversibly interconnectable with (i) at least some of the stationary hospital services and (ii) with the receptacle; thereby ensuring that the patient is uninterruptedly connected to the LSU before, during and after inserting into the RISF.

16. A method of magnetic resonance imaging (MRI) a neonate demanding an intensive care; the method comprising the steps of:
a. providing an autonomous cradle configured for life supporting a premature neonate during magnetic resonance imaging procedure; the cradle comprising a receptacle configured for receiving the neonate and inserting into a MRI apparatus; the receptacle being magnetically transparent;
b. placing the neonate into the receptacle;
c. supporting neonate life performed by a portable unit of neonate life support unit (LSU) remotely connected to the receptacle such that a magnetic field of the MRI apparatus is not disturbed by the life support unit;
d. inserting the receptacle into the MRI apparatus; and
e. performing MRI scan;
wherein the LSU is reversibly interconnectable with (i) at least some of the stationary hospital services and (ii) with the receptacle; thereby ensuring that the neonate is uninterruptedly connected to the LSU before, during and after inserting into an MRI apparatus transfer to the MRI.

17. The method according to claim 16, wherein the life support unit is either electric grid independent or battery energized.

18. The method according to claim 16, wherein at least one of the following is being held true (a) the step of supporting neonate life comprises administering maintenance media into the neonate; (b) a breathing gas and the maintenance media are fed to the neonate by means of magnetically transparent feeding pipes; (c) the receptacle is provided with a connection interface device configured for interconnecting feeding pipes within the receptacle with the life support unit; (d) the step of supporting neonate life comprises artificial pulmonary ventilation of the neonate; and any combination thereof.

19. A method of stabilizing life supports service lines (LSSLs) to a patient in need of intensive care; the method comprising the steps of:
a. providing an autonomous cradle configured for life supporting a patient during treating in an RISF; the cradle comprising a receptacle configured for receiving the patient and inserting into the RISF; the receptacle being adapted to be compatible with the RISF;
b. placing the patient into the receptacle;
c. providing patient's life support by means of a portable LSU; remotely connecting the receptacle and LSSLs and immobilizing the same;
d. inserting the receptacle into the RISF; and
e. performing the treatment;
wherein the LSU is reversibly interconnectable with (i) at least some of the stationary hospital services and (ii) with the receptacle; thereby ensuring that the patient is uninterruptedly connected to the LSU before, during and after inserting into the RISF; and
further wherein at patient's end the LSSLs are immobilized thereby ensuring minimum trauma to the patient airways, blood vessels, circulatory system and/or gastroenterological tracts.

20. The method of claim 19, wherein the step of remotely connecting the receptacle and LSSLs and immobilizing the same further comprising step of providing an LSSL-interface with LSSLs inlets on the exterior side of the interface and inside LSSL outlets on the interior side of the interface.
